(19) United States
(12) Patent Application Publication
(10) Pub. No.: US 2004/0048828 A1
(43) Pub. Date: Mar. 11, 2004

(54) Immunosuppressive agent and food

(75) Inventors: Nobukazu Onishi, Hiroshima-shi (JP); Kunihiko Hashimoto, Hiroshima-shi (JP); Hisao Shimizu, Mihara-shi (JP)

Correspondence Address: SUGHRUE MION, PLLC, 2100 PENNSYLVANIA AVENUE, N.W., WASHINGTON, DC, 20037, US

(73) Assignee: The Japan Research and Development Association for New Functional Foods

(21) Appl. No.: 10/395,274

(22) Filed: Mar. 25, 2003

(30) Foreign Application Priority Data
Sep. 9, 2002 (JP) P.2002-263067

(51) Int. Cl. A61K 31/736

(52) U.S. Cl. 514/54

(57) ABSTRACT

An immunosuppressive agent and food, which can prevent type I allergic diseases, autoimmune diseases, rejections in organ transplant and improve morbid states even when these diseases are induced, and which are safe and easy to intake. The immunosuppressive agent and food contain a fine powder of a hydrophilic polysaccharide. Preferably, the fine powder of hydrophilic polysaccharide is in the form of glucomannan which is refined konjak flour, has a dietary fiber content of 90% or more, and is easily soluble in water. For making the glucomannan into an easily water-soluble state, it is preferably subjected to a pulverization treatment. Preferably, the glucomannan subjected to the pulverization treatment has a weight average molecular weight of 1,000,000 or more and an average particle diameter of 100 μm or less, and a period of time until 1% aqueous solution thereof reaches the viscosity peak at room temperature is within 30 minutes. As a fort, powder, capsule, tablet, pill or granule may be mentioned.

### FIELD OF THE INVENTION

The present invention relates to an immunosuppressive agent and food from which preventive and improving actions of type I allergic diseases, autoimmune diseases, rejections in organ transplant can be expected. Furthermore, the invention relates to an immunosuppressive animal to which the immunosuppressive agent is administered and a process for producing the same.

### BACKGROUND OF THE INVENTION

Since suppression of immune response is necessary for prevention and treatment of allergic diseases induced by an excessive reaction of immune system against a foreign antibody, autoimmune diseases such as chronic rheumatism which induces tissue injury by an autoantibody, and rejections in organ transplant and the like, immunosuppressive agents are widely used clinically. However, currently employed steroid agents and pharmaceutical agents acting on nucleic acid-synthesizing system have a wide variety of actions and induce a large number of serious side effects. Moreover, Cyclosporine A and Tacrolimus (FX506) have a strong suppressive action but it is difficult to employ them for a long period of time because of problems of renal toxicity and hepatic toxicity

### SUMMARY OF THE INVENTION

Taking the aforementioned related arts into consideration, the invention is to provide an immunosuppressive agent and food, which can suppress the excessive immune response in the living body and treat, prevent and/or improve autoimmune diseases such as chronic rheumatism, multiple sclerosis and systemic lupus erythematosus, rejections in organ transplant and the like, and which are safe and easy to intake.

The present inventors have already found that purified glucomannan (dietary fiber content of 90% or more) which is obtained from refined konjak flour by an ethanol precipitation method and which is further made into easily water soluble state by a pulverization treatment (average particle diameter of 100 μm or less) or the like treatment has a markedly high IgE antibody inhibitory ability as compared with refined konjak flour and a function to prevent allergic diseases (Japanese Patent Application No. 2001-237993). In the successive studies, they have for the first time found that the substance has suppressive action of production of IgG antibody in sera, suppressive action of spleen T-cell growth and suppressive action of cytokine production as well as suppressive action of antigen-specific antibody production and suppressive action of cell growth in mixed lymphocyte reaction. The present invention has been accomplished based on this finding.

That is, the invention relates to the following.

(1) An immunosuppressive agent containing a fine powder of a hydrophilic polysaccharide,

(2) The immunosuppressive agent described in the above item (1), wherein the fine powder of hydrophilic polysaccharide is in the form of purified glucomannan subjected to a pulverization treatment to have an average particle diameter of 100 μm or less, the purified glucomannan being obtained from refined konjak flour by an ethanol precipitation method and having a dietary fiber content of 90% or more.

(3) The immunosuppressive agent described in the above item (1) or (2), wherein a period of time until 1% aqueous solution of the fine powder of hydrophilic polysaccharide reaches the viscosity peak at room temperature is within 30 minutes.

(4) The immunosuppressive agent described in any one of the above items (1) to (3), having a form of powder, capsule, tablet, pill or granule.

(5) An immunosuppressive animal wherein the immunosuppressive agent described in any one of the above items (1) to (4) is administered to the animal.

(6) A process for making an immunosuppressive animal comprising a step of administering the immunosuppressive agent described in any one of the above items (1) to (4) to the animal.

(7) An immunosuppressive food containing a fine powder of a hydrophilic polysaccharide.

(8) The immunosuppressive food described in the above item (7), wherein the fine powder of hydrophilic polysaccharide is in the form of purified glucomannan subjected to a pulverization treatment to have an average particle diameter of 100 μm or less, the purified glucomannan being obtained from refined konjak flour by an ethanol precipitation method and having a dietary fiber content of 90% or more.

(9) The immunosuppressive food described in the above item (7) or (8), wherein a period of time until 1% aqueous solution of the fine powder of hydrophilic polysaccharide reaches the viscosity peak at room temperature is within 30 minutes.

(10) The immunosuppressive food described in any one of the above items (7) to (9), having a form of powder, capsule, tablet, pill or granule.

(11) An immunosuppressive animal wherein the immunosuppressive food described in any one of the above items (7) to (10) is administered to the animal.

(12) A process for making an immunosuppressive animal comprising a step of administering the immunosuppressive food described in any one of the above items (7) to (10) to the animal.

In the above respective subject matter, preferably, inhibition of IgE antibody is excluded.

### BRIEF DESCRIPTION OF THE DRAWINGS

By way of example and to make the description more clear, reference is made to the accompanying drawing in which:

FIG. 1 is a graph showing a result of Example 1, which shows influence of the finely pulverized purified glucomannan on total IgG1 and IG2a contents.

FIG. 2 is a graph showing a result of Example 2, which shows influence of the finely pulverized purified glucomannan on growth potency of spleen T-cells.

FIG. 3 is a graph showing a result of Example 2, which shows influence of the finely pulverized purified glucomannan on cytokine productivity of spleen T-cells.

FIG. 4 is a graph showing a result of Example 3, which shows influence of the finely pulverized purified glucomannan on OVA-specific IgG1 antibody value.

FIG. 5 is a graph showing a result of Example 4, which shows influence of the finely pulverized purified glucomannan on growth potency of T-cells in rat mixed lymphocyte reaction.

FIG. 6 is a graph showing a result of Example 4, which shows influence of the finely pulverized purified glucomannan on IL-2 production in rat mixed lymphocyte reaction.

DETAILED DESCRIPTION OF THE INVENTION

The subject to be treated by the present invention include vertebrates, preferably warm-blooded animals, more preferably mammals, and further more preferably human. The immunosuppressive animal of the present invention is not particularly limited and includes non-human animals such as mouse, rat, rabbit, goat, cat, dog, bovine, horse, and the like.

The fine powder of hydrophilic polysaccharide which is a main component of the immunosuppressive agent and food of the invention has a long period of actually used results an a food material and a food additive as glucomannan particularly in Japan and also has a high safety. According to the present invention, its continuous internal use can suppress excessive response of immune system and prevent autoimmune diseases, rejections in organ transplant, and the like.

The invention relates to a specific use of the fine powder of hydrophilic polysaccharide. Though materials of the fine powder of hydrophilic polysaccharide to be used in the invention are not particularly limited, refined konjak flour and the like refined from konjak tuberous roots and the like are desirable from the viewpoint of easy availability. The refined konjak flour to be used in the invention is described in detail in “konjak no Kagaku (Science of konjak) (established in 1993)” edited by Satoshi Okimasu. The term “konjak” as used herein means Amorphophallus konjac, which has been eaten as food, especially in Japan, and which may be called as “devil's tongue”.

In the invention, the fine powder of hydrophilic polysaccharide is easily soluble in water. Though the method for making the fine powder of hydrophilic polysaccharide into easily water-soluble state is not particularly limited, a pulverization treatment is preferable from the viewpoint of easy workability. It is preferable that the fine powder of hydrophilic polysaccharide made into easily water-soluble state by the pulverization treatment as described above has an average particle diameter of 100 micrometer or less. More preferably, a period of time until its 1% aqueous solution reaches the viscosity peak at room temperature is within 30 minutes. Furthermore, a weight average molecular weight thereof is preferably 1,000,000 or more.

Moreover, as the aforementioned fine powder of hydrophilic polysaccharide, glucomannan is preferably used. More preferably, purified glucomannan having a dietary fiber content of 90% or more is used. The method for controlling the dietary fiber content within the above range is not particularly limited but it is preferable to obtain purified glucomannan by purifying the aforementioned refined konjak flour by an ethanol precipitation method.

The immunosuppressive agent and food of the invention may be embodied in any form of powders, capsules such as gelatin capsules, tablets, pills or granules. Also, it may be used together with a filler or may contain other auxiliary components so long as they do not impair functions of the immunosuppressive agent. Any substance which is harmless to human can be used as the auxiliary component which may be contained. Intake of the immunosuppressive agent is effective generally at an oral dose of from 1 to 50 g/60 kg-body weight per day, preferably from 5 to 50 g/60 kg-body weight per day, in terms of the amount of the fine powder of hydrophilic polysaccharide. The dose that showed good results in mice corresponds to about 5 g/60 kg-body weight per day in human. The dose is preferably less than 10 g/60 kg-body weight per day in order to avoid diarrhea which might occur in a higher dose depending on the individual. In view of the effects of the invention, the immunosuppressive agent is preferably taken continuously.

In addition, it may be embodied also in a form in which it is contained in general food, namely as an immunosuppressive food. The food is not particularly limited. In order to obtain an immunosuppressive food, the purified glucomannan of the invention may be blended in response to the property of respective food, for example in a powdery form for a biscuit-like food. Its minimum concentration in food effective in exerting the effects of the invention is 1% by weight or more in terms of the amount of the fine powder of hydrophilic polysaccharide.

The invention will be illustrated in greater detail with reference to the following Examples, but the invention should not be construed as being limited thereto.

### Example 1

Suppression of IgG Antibody in Sera

As the animal to be tested, a spontaneously induced atopic dermatitis model animal NC/Nga mouse (hereinafter referred to as “NC mouse”) [Matsuda H et al.; Int. Immunol., 9, 461 (1997)] was used. Males of 4 weeks of age were purchased from Japan SLC. Feeding MF (solid feed) manufactured by Oriental Yeast, Co., Ltd was used as a basal feed. Respective test feed was used by adding 5% by weight of each of the following additives manufactured by Shimizu Kagaku to the basal feed.

The additives to be added to the respective feed are shown below.

Test feed 1: refined konjak flour (KP; average particle diameter, about 300 μm)

Test feed 2: purified high purity glucomannan having the dietary fiber content of 99% or more (PA; average particle diameter, about 300 μm)

Test feed 3: finely pulverized purified glucomannan made into easily water-soluble state by applying a pulverization treatment (S; average particle diameter, about 100 μm)

Test feed 4: finely pulverized purified glucomannan made into easily water-soluble state by applying a pulverization treatment (Z; average particle diameter, about 75 μm)

Test feed 5: granulated product of finely pulverized purified glucomannan S (S-gw; average particle diameter, about 150 μa)

Using 5 animals of the NC mouse as one group, each group was allowed to feed on the basal feed or each test feed freely for 8 weeks and then blood samples were collected. The blood samples were centrifuged at 1,700 rpm for 10 minutes to obtain sera.

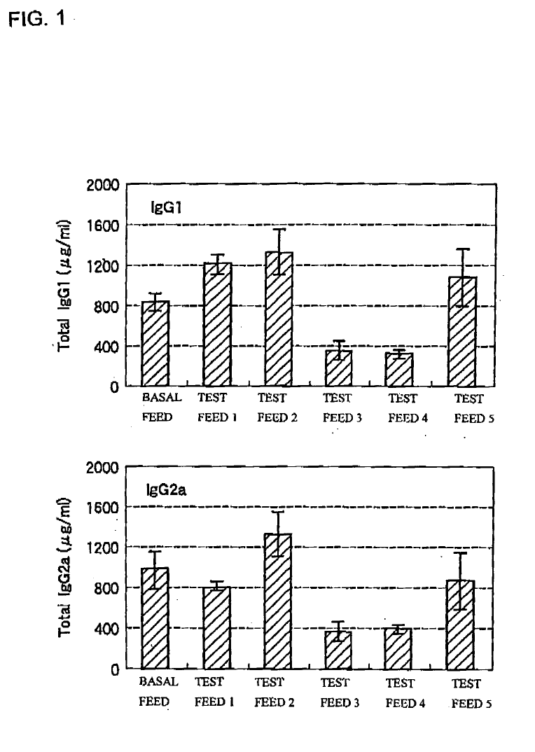

Total IgG1 and IgG2a contents in the thus obtained sera were analyzed by the sandwich ELISA method. Results are shown in FIG. 1.

As is apparent from FIG. 1, with regard to the total IgG1 and IgG2a contents at the time of 12 weeks of age, no difference was observed in the refined konjak flour (test feed 1) administered group, in the purified high purity glucomannan (test feed 2) administered group, and in the granulated product of finely pulverized purified glucomannan (test feed 5) administered group, but remarkable decrease was confirmed in the finely pulverized purified glucomannan (test feed 3 and 4) administered groups.

### EXAMPLE 2

Suppression of Growth of Spleen T-Cells and Suppression of Cytokine Production

Each test animal group prepared in Example 1 was allowed to feed on the basal feed or each test feed freely for 8 weeks and then spleen cells were collected from each NC mouse in the basal feed group and the finely pulverized purified glucomannan groups (the test feed 3 and 4 groups). Erythrocytes were removed from the spleen cells by a lysis buffer (150 mM NH₄Cl, 15 mM NaHCO₃, 0.1 mM EDTA 2Na [pH 7.33]). The resulting cells were washed with PBS and then suspended into RPMI-1640 medium incorporated with 10% FCS. Thereafter, number of the living cells stained with trypan blue was counted and adjusted to 2×10⁶ cells/ml.

A 96-well plate in which anti-mouse CD3 antibody was solid-layered was washed with RPMI-1640 medium and then 50 μl of anti-mouse CD28 antibody (1 μg/ml) was added to each well As a control for growth measurement, a well incorporated with 50 μl of 10% FCS-RPMI-1640 medium was prepared on each plate. Thereinto was inoculated 50 μl of the adjusted cell suspension, followed by 3 days (72 hours) of culturing at 37° C. under the condition of 5% CO₂.

Growth potency was investigated by measuring intake of BrdU (5-Bromo-2′-deoxy-uridine) by the cells, the BrdU being added 15 hours before termination of cell culturing. For the measurement, BrdU Labeling and Detection Kit III (Roche Molecular Biochemicals, Mannheim, Germany) was used.

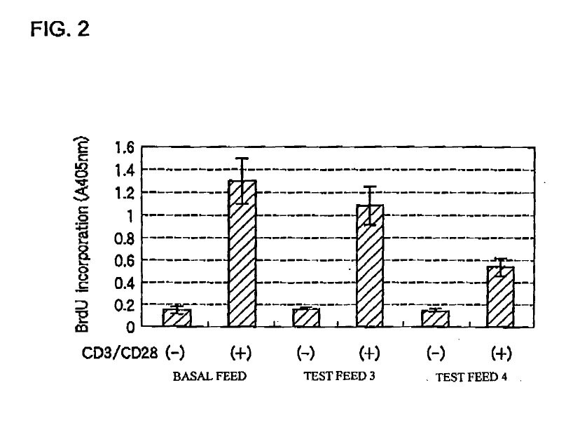

After 3 days of culturing, the culture solution was transferred into another plate, centrifuged at 300 rpm for 10 minutes, and then dried at 60° C. until the medium was removed. Thereafter, the cells were fixed to the plate with a fixing solution (70 M ethanol, 0.5% HCl) at −20° C. for 30 minutes. Subsequently, DNA was partly digested with a nuclease solution and then allowed to react with anti-BrdU-POD antibody at 37° C. for 30 minutes. Then, ABTS, a substrate of peroxidase was added and absorbance at 405 nm was measured on a plate reader. FIG. 2 shows results of the above investigation of influence of the finely pulverized purified glucomannan on growth potency of spleen T-cells of the mouse at the time of 12-weeks of age.

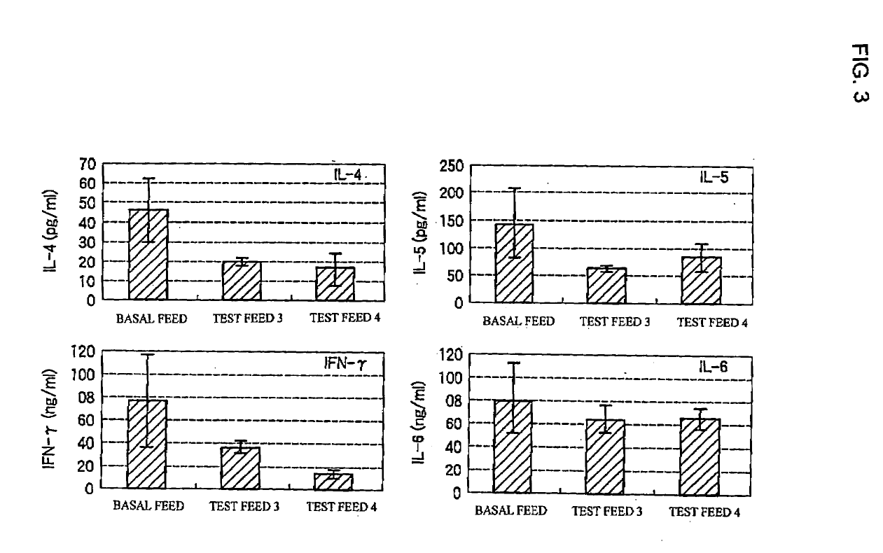

The spleen cells and Payer patch cells were cultured for 3 days and then the culture solution was transferred into a conical tube. The cells were removed by centrifugation at 1500 rpm for 5 minutes, and cytokine production (IL-4, IL-5, IL-6, IFN-γ) in the culture supernatant was measured by the sandwich ELISA method. FIG. 3 shows results of the influence of the finely pulverized purified glucomannan on cytokine productivity thus measured.

As a result, the growth potency of T-cells by stimulation with CD3 and CD28 decreased in the finely pulverized purified glucomannan (test feed 3 and 4) administered groups as compared with the basal teed administered group. The decrease of the growth potency is more remarkable in the test feed 4 administered group, the feed having a small particle diameter of glucomannan. As is apparent from FIG. 3, with regard to the cytokine production of T-cells, the production of IL-4, IL-5, IL-6 and IFN-γ decreased in the finely pulverized purified glucomannan (test feed 3 and 4) administered groups as compared with the basal feed administered group.

### EXAMPLE 3

Suppression of Antigen-Specific Antibody Value

Using 3 animals of male ICR mouse (Japan SLC) of 4 weeks of age as one group, each group was allowed to feed on a feed and water freely at 23±3° C. for 2 weeks at light-dark cycle every 12 hours. With regard to the feed, the investigation was carried out in the basal feed group or in the finely pulverized purified glucomannan (test feed 4) group. On the next day after 5 days of feeding, 100 μl of an antigen Alum suspension was administered into the abdominal cavity and, after 6 days therefrom, additional immunization was carried out similarly.

As the antigen Alum suspension to be used was prepared by adding 80 μl of ovalbumin (OVA) solution (in 0.9% NaCl solution) adjusted to 10 mg/ml to 20 μl of Alum (in 0.9% NaCl solution) adjusted to 20 mg/ml and stirring them thoroughly.

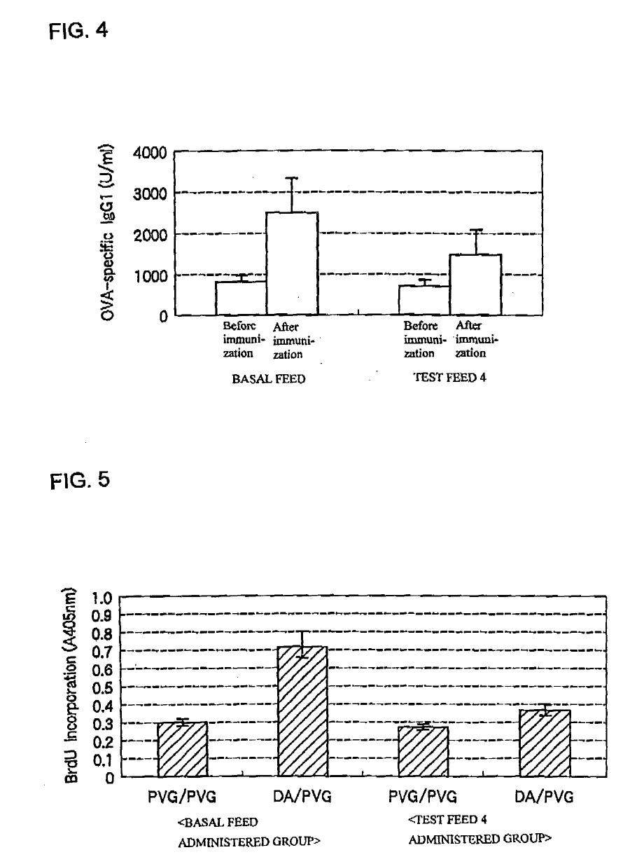

Blood samples were collected on the day at which feeding was started and 3 days after the additional immunization, and OVA-specific IgG1 antibody value was analyzed by the ELISA method. Results were shown in FIG. 4.

As is apparent from FIG. 4, increase of OVA-specific IgG1 antibody value reduced in the finely pulverized purified glucomannan (test feed 4) administered groups as compared with the basal feed administered group.

### Example 4

Suppression of Mixed Lymphocyte Reaction (MLR) Preparation of Reactive Cells

PVG rat was used as the animal to be tested. As the PVG rat, males of 4 weeks of age were purchased from Japan SLC. Feeding MF (solid feed) manufactured by Oriental Yeast, Co., Ltd was used as a basal feed. Test feed 4 used in Example 1 was used as a test feed. Using 3 animals of the PVG rat as one group, each group was allowed to feed on each feed and water freely at 23±3° C. at light-dark cycle every 12 hours. After 6 weeks of feeding, each spleen was collected. Erythrocytes were removed from the spleen cells by a lysis buffer (150 mM NH₄Cl, 15 mM NaHCO₃, 0.1 mM EDTA 2Na[pH 7.3]). The resulting spleen cells were washed twice with PBS and then suspended into RPMI-1640 medium incorporated with 100 U/ml penicillin, 100 mg/ml streptomycin, 50 mM 2-mercaptoethanol, and 10% fetal calf serum (FCS, mfd. by SIGMA). Thereafter, number of the living cells stained with trypan blue was counted and adjusted to 5×10⁵ cells/ml with RPMI-1640 medium incorporated with 10% FCS (10% FCS-RPMI medium). Adjustment of stimopxulant cells

Spleen cells were collected from DA rats allowed to feed on the basal feed as in the case of PVG rats, and the growth potency was deprived by an MMC treatment shown below.

The collected spleen cells were adjusted to 1×10⁷ cells/ml with a medium wherein 2 mg of mitomycin C (Kyowa Hakko Kogyo Co., Ltd.) was adjusted to 25 mg/ml with 10% FCS-RPMI medium. Then, the cells were incubated for 30 minutes in a CO₂ incubator and then subjected to centrifugal washing three times with RPMI-1640 medium. Number of the living cells of the stimulant cells stained with trypan blue was counted and adjusted to 8×10⁶ cells/ml with 10% FCS-RPMI medium. Mixed lymphocyte reaction (MLR)

A 100 μl portion of each of the stimulant cells (DA: 8×10⁶ cells/ml) and the reactive cells (PVG: 5×10⁵ cells/ml) was added to a 96 well round-bottom plate (mfd. by Nunc), followed by 3.5 days (84 hours) of culturing at 37° C. under the condition of 5% CO₂/95% air. As a negative control of cell growth, after washing with—1640 medium, 50 μl of anti-mouse CD 28 antibody (1 μg/ml) was added to each well. Moreover, as a control for growth measurement, spleen cells of OVG rats treated with MMC and the reactive cells (OVG spleen cells) were subjected to mixed culture.

Growth potency was investigated by measuring intake of BrdU (5-Bromo-2′-deoxy-uridine) by the cells, the BrdU being added 15 hours before termination of cell culturing. For the measurement, BrdU Labeling and Detection Kit III (Roche Molecular Biochemicals, Mannheim, Germany) was used.

After 3.5 days (84 hours) of culturing, the culture solution was transferred into another plate, centrifuged at 300 rpm for 10 minutes, and then dried at 60° C. until the medium was removed. Thereafter, the cells were fixed to the plate with a fixing solution (70 M ethanol, 0.5% RCl) at −20° C. for 30 minutes. Subsequently, DNA was partly digested with a nuclease solution and then allowed to react with anti-BrdU-POD antibody at 37° C. for 30 minutes. Then, ABTS, a substrate of peroxidase was added and absorbance at 405 nm was measured on a plate reader. Results are shown in FIG. 5.

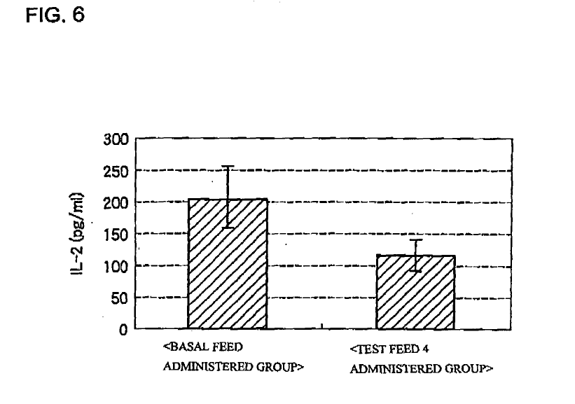

After 3 days of mixed lymphocyte culturing, the culture solution was transferred into a conical tube. The cells were removed by centrifugation at 1500 rpm for 5 minutes, and IL-2 production in the culture supernatant was measured by the sandwich ELISA method. Results are shown in FIG. 6.

As is apparent from FIG. 5, a remarkable suppressive effect on cell growth potency in the mixed lymphocyte reaction (MLR) was observed in the finely pulverized purified glucomannan (test feed 4) administered group as compared with the basal feed administered group. Furthermore, as is also apparent from FIG. 6, a remarkable suppressive effect on IL-2 production in the mixed lymphocyte reaction (MLR) was observed in the finely pulverized purified glucomannan (test feed 4) administered group as compared with the basal feed administered group.

As described above, according to the invention, the immunosuppressive agent and food containing the fine powder of hydrophilic polysaccharide can suppress an excessive immune response in the living body, and prevent and improve type I allergic diseases, autoimmune one diseases such as chronic rheumatism, multiple sclerosis and systemic lupus erythematosus, rejections in organ transplant, and the like.

While the invention has been described in detail and with reference to specific embodiments thereof, it will be apparent to one skilled in the art that various changes and modifications can be made therein without departing from the scope thereof.

This application is based on Japanese patent application No. 2002-263067 filed Mar. 25, 2002, the entire contents thereof being hereby incorporated by reference.

What is clamed is:

1. An immunosuppressive agent comprising a fine powder of a hydrophilic polysaccharide.

2. The immunosuppressive agent according to claim 1, wherein the fine powder of hydrophilic polysaccharide is in the form of purified glucomannan subjected to a pulverization treatment to have an average particle diameter of 100 μm or less, the purified glucomannan being obtained from refined konjak flour by an ethanol precipitation method and having a dietary fiber content of 90% or more.

3. The immunosuppressive agent according to claim 1, wherein a period of time until 1% aqueous solution of the fine powder of hydrophilic polysaccharide reaches the viscosity peak at room temperature is within 30 minutes.

4. The immunosuppressive agent according to claim 1, which is in a form of powder, capsule, tablet, pill or granule.

5. An immunosuppressive animal wherein the imaunosuppressive agent according to any one of claims 1 to 4 has been administered to the animal.

6. A process for producing an immunosuppressive animal comprising a step of administering the immunosuppressive agent according to any one of claims 1 to 4 to the animal.

7. An immunosuppressive food comprising a fine powder of a hydrophilic polysaccharide.

8. The immunosuppressive food according to claim 7, wherein the fine powder of hydrophilic polysaccharide is in the form of purified glucomannan subjected to a pulverization treatment to have an average particle diameter of 100 μm or less, the purified glucomannan being obtained from refined konjak flour by an ethanol precipitation method and having a dietary fiber content of 90% or more.

9. The immunosuppressive food according to claim 7, wherein a period of time until 1% aqueous solution of the fine powder of hydrophilic polysaccharide reaches the viscosity peak at room temperature is within 30 minutes.

10. The immunosuppressive food according to claim 7, having a form of powder, capsule, tablet, pill or granule.

11. An immunosuppressive animal wherein the immunosuppressive food according to claim 7 has been administered to the animal.

12. A process for making an immunosuppressive animal comprising a step of administering the immunosuppressive food according to claim 7 to the animal.
